(19) United States
(12) Patent Application Publication
(10) Pub. No.: US 2017/0348157 A1
(43) Pub. Date: Dec. 7, 2017

(54) PARTICLE ENTRAINED AIR-PERMEABLE STRUCTURES

(71) Applicant: Kim PATCHETT, Essex (GB)

(72) Inventor: Kim PATCHETT, Essex (GB)

(21) Appl. No.: 15/539,470

(22) PCT Filed: Sep. 23, 2015
(86) PCT No.: PCT/GB2015/052746

§ 371 Date: Jun. 23, 2017

(30) Foreign Application Priority Data
Dec. 28, 2014 (GB) 1423274.8

(51) Int. Cl. A61F 13/15; B32B 5/02; B32B 37/08; B32B 37/10

(52) U.S. Cl. CPC A61F 13/15658; B32B 37/1018; B32B 5/022; B32B 37/08; A61F 13/15699; B32B 2307/728; B32B 2310/028; B32B 2555/02

(57) ABSTRACT

A method is provided, for dissipating and entrapping super absorbent polymer particles (11, 12, 13, 14) within air-permeable, non-woven structures (100), for use in the construction of absorbent articles (600). The method comprises the steps of: (i) of constructing an air-permeable, non-woven structure (100) comprising at least first (1), second (2) and third (3) layers of non-woven fabric, each said layer having void spaces of differing size defined therein; (ii) dispersing absorbent particles (11, 12, 13, 4) onto an external surface (10) of the highest numbered layer of said air-permeable, non-woven structure (100) formed in step (i); and (iii) dissipating the dispersed absorbent particles (11, 12, 13, 14) within the air-permeable, non-woven structure (100) by applying an external energy source acting upon the absorbent particles (11, 12, 13, 14) in a direction substantially normal to the plane of the external surface (10) of the air-permeable, non-woven structure (100).

This invention relates to a method for dissipating and entrapping absorbent particles within air-permeable, non-woven structures. In particular, it relates to such a method for dissipating and entrapping particles that in their manufactured or synthesized state have a mean particle diameter of between about 10 μm and 1000 μm. It will be noted however that the invention may be similarly practiced with particles having a mean diameter smaller than 10 μm and larger than 1000 μm.

Absorbent particles suitable for processing according to the method of the present invention include but, are not limited to: sodium polyacrylate, polyacrylamide copolymer, ethylene maleic anhydride copolymer, cross-linked carboxymethyl-cellulose, polyvinyl alcohol copolymers, cross-linked polyethylene oxide, and starch grafted copolymers of polyacrylonitrile, either solely, or in combination.

In recent years, there has been a trend in the hygiene industry to reduce the mass of fluff pulp contained within hygiene products such as infant diapers, adult incontinence products and feminine hygiene products. The trend to reduce the mass of fluff pulp within hygiene products is directed at using less mass of raw materials in the end product thereby both reducing the manufacturing demand for raw materials, and reducing the amount of soiled materials for disposal after the product has been used for its intended purpose. A further trend to reduce the mass of fluff pulp with hygiene products is directed at reducing or eliminating the atmospheric dust within end product manufacturing plants emanating from the processing of the fluff pulp.

The conventional structure of, for example, an infant diaper has at its core a combined mass of fluff pulp and absorbent particles known as SAP (super absorbent polymers). Conventional methods for combining fluff pulp and SAP result in the SAP being randomly distributed throughout the mass of the fluff pulp core. Generally, the SAP comprises sodium polyacrylate, which may be acquired from a number of commercial sources. Although each commercially available grade of sodium polyacrylate will have a different particle size distribution (PSD) when compared to another grade or a different manufacturer, a typical PSD is shown below:

Airborne particles have irregular shapes, and their aerodynamic behaviour is expressed in terms of the diameter of an idealised spherical particle known as the aerodynamic diameter. Particles are sampled and described on the basis of their aerodynamic diameter, which is usually simply referred to as particle size. Particles having the same aerodynamic diameter may have different dimensions and shapes. The aerodynamic diameter of an irregular particle is defined as the diameter of the spherical particle with a density of 1000 kg m⁻³ and the same settling velocity as the irregular particle.

It is well understood that particles having an aerodynamic diameter of between 0.5 μm and 10 μm may easily settle within the transfer region of the human lungs (the Alveoli) and may lead to acute or chronic ill health effects depending upon the type of powder and its specific chemical and physical composition. Particles of aerodynamic diameter in the range of 0.5 μm and 10 μm are defined as being within the respirable range. Particles of a size below the respirable range may be exhaled naturally after entering the lungs. Particles of a size above the respirable range are removed by the impingement of nasal hairs and very fine hairs (Cilia) that line the bronchi and trachea and trap foreign bodies within the respiratory system. Trapped foreign bodies are covered in mucus and passed out up into the throat where they are swallowed, sneezed or spat out. Mucus ensures that particles do not become re-entrained in the inhalation flow and ultimately particles are discharged from the body thereby protecting the lungs from particle ingress.

In the United Kingdom, the threshold limit value (TLV) applied to particles within the respirable range have been published by the Health and Safety Executive (HSE). The Control of Substances Hazardous to Health (COSHH) Act 1988, introduced the Maximum Exposure Limit (MEL) and the Occupational Exposure Standard (OES).

Historic TLV values and the level of hazard that they pose are as follows:

Group 1—very dangerous, 0 μg m⁻³ to 50 μg m⁻³. Readily give rise to fibrosis and includes: beryllium, silica in the cristobalite form and blue asbestos (5 fibres/cm³ and less than 5 μm in length). Note that asbestos is not classified using aerodynamic diameter due to its unusual fibre like shape;

Group 2—dangerous, 50 μg m⁻³ to 250 μg m⁻³. Includes: asbestos (other forms of and with 5 fibres/cm³>5 μm in length), silica such as quartz and mixed powders containing 20% or more of silica;

Group 3—moderate risk, 250 μg m⁻³ to 1000 μg m⁻³. Includes: mixed powders of <20% silica, talc, mica, kaolin, cotton, organic dust, graphite and coal; and

Group 4—low risk, >1 mg m⁻³, includes: cement powder, limestone, glass, barites, perlite, iron oxide, magnesia and zinc oxide.

Of course, sensible precautions such as the wearing of suitable dust masks within the working environment may protect against the ingress of particles within the respirable range into the lungs but in addition to the human health hazards associated with processing small particles, it may also be the case that contamination of the end product in manufacture or the production machinery itself may be problematic.

Consequently, it is desirable to create a process for eliminating fluff pulp and other potential dust hazards, and in particular those particles that fall within the respirable range of between 0.5 μm and 10 μm.

Coincidentally, in the case of infant diapers, adult incontinence products and feminine hygiene products, a reduction in the mass of fluff pulp within the product also increases the number of finished and packaged products that can be transported within a given volume and therefore reduces transport costs per product unit shipped. This therefore has a beneficial impact on both reducing air pollution and reducing carbon di-oxide emissions from burning fossil fuels during transport.

It would therefore be beneficial if fluff pulp could be completely eliminated from within the core structure of infant diapers, adult incontinence products and feminine hygiene products. It would be further beneficial if the SAP were distributed within a fluff pulp free, air-permeable core in a controlled manner, such that the different sizes of particles within a specific grade of SAP were positioned to absorb liquid waste in the most efficient manner rather than, as is currently the case, where the different sizes of SAP particles within a specific grade are randomly distributed throughout the core.

The present invention therefore seeks to provide a method for managing absorbent particles within a fluff pulp free, air-permeable, non-woven absorbent structure, for use within infant diaper, adult incontinence products, feminine hygiene and other hygiene and healthcare products. The present invention further seeks to manage the distribution of said particles in a controlled manner, such that the distribution of said particles within the air-permeable, non-woven structure is predetermined by particle size, thereby to provide the most efficient mechanism for absorbing liquid waste as is feasible.

Hygiene products as referred to herein include, but are not limited to, infant diapers, adult incontinence products and feminine hygiene products. When in use by the consumer, each of these products is designed to manage and absorb liquids expelled from the user's body in the form of urine, liquid faeces and menstrual fluids individually or in combination.

Since the mid 1970's the inclusion of Super Absorbent Polymers (SAP) within infant diapers has been common practice. SAPs such as for example, sodium polyacrylate have usually been incorporated into the structure of infant diapers by blending the polymer particles into fluff pulp, to form an absorbent core of the diaper. Similar structures are also incorporated within adult incontinence and feminine hygiene products. Since the SAPs are only blended within the fluff pulp core it is necessary to take steps to stop the SAP particles from leaching out of the fluff pulp core and making contact with the skin of, for example, an infant wearer of the diaper. Although SAPs are considered harmless to human skin, it is not desirable to contaminate an infant's skin with SAP particles.

Conventional methods for preventing SAP from leaching out of the fluff pulp diaper core include attaching lightweight layers of additional non-woven fabric and or polymer film onto both the surface and the edges of the fluff pulp core. Within the hygiene industry, this is often referred to as core wrap. This method of preventing SAP from leaching out of the fluff pulp core has been shown to be fallible and it is not uncommon for small amounts of SAP to leach out of the fluff pulp core and onto the infant or other wearer's skin.

It would therefore be desirable to eliminate fluff pulp entirely from the core of all hygiene products including infant diapers, adult incontinence products and feminine hygiene products thereby reducing the mass and thickness of the manufactured products whilst maintaining the ability of the products to absorb the required volumes of liquid waste whether it is urine, liquid faeces or menstrual fluids or any combination of these liquids whilst at the same time retaining the SAP particles fully within the core structure of the product.

The present invention provides a method for incorporating absorbent particles within hygiene products in such a way that the particles are distributed within the air-permeable, non-woven structure of the product in a controlled and pre-determined manner such that the particles are able to absorb liquid waste in the most efficient manner feasible whilst also being fully retained within the structure of the product such that the particles are unable to leach out onto the wearers skin.

According to the present invention there is provided a method for dissipating and entrapping absorbent particles within air permeable structures, for use in the construction of absorbent articles, said method comprising the steps of:

(i) constructing an air-permeable, non-woven structure comprising at least first, second and third layers of non-woven fabric, each said numbered layer (n) being bonded, manufactured onto or otherwise joined to each subsequently numbered layer (n+1), and wherein fibres in each said layer are arranged so as to define void spaces therebetween of pre-determined size, corresponding to a given absorbent particle size distribution range;

and wherein the void spaces in each said numbered layer (n), are of smaller size than the void spaces in each subsequently numbered layer (n+1);

(ii) dispersing, by controllable mechanical means, absorbent particles onto an external surface of the highest numbered layer of said air-permeable, non-woven structure formed in step (i), said absorbent particles having a pre-determined particle size distribution range;

(iii) dissipating said dispersed absorbent particles within the air-permeable, non-woven structure by applying an external energy source acting upon the absorbent particles in a direction substantially normal to the plane of the external surface of the air-permeable, non-woven structure.

In certain embodiments of the present invention, the method may comprise the additional further steps of:

(iv) subjecting at least one layer of the air-permeable, non-woven structure to an external heat source, and subsequently effecting or allowing cooling of said at least one layer, so as to entrap said absorbent particles within said air-permeable, non-woven structure; and/or

(v) welding, bonding or otherwise attaching a further layer to the external surface of the highest numbered layer of said air-permeable, non-woven structure incorporating said dissipated absorbent particles.

The first layer, as referred to in step (i), should be a hydrophilic or hydrophobic fibre layer constructed such that the spaces or voids between the individual fibres that constitute the first layer are too small for a first size of the particle size distribution (PSD) of a given grade of the absorbent particle types to be incorporated within the air-permeable, non-woven structure to pass through. Within the hygiene industry this layer is often referred to as the acquisition and distribution layer (ADL).

The second layer, attached, manufactured onto or otherwise fixed to the first layer, should comprise a second hydrophilic or hydrophobic fibre layer such that said first size of given grade of absorbent particles may pass into the spaces and or voids between the individual fibres that constitute the second layer but the same particles are unable to pass into or through the spaces or voids in the first described layer or ADL.

The third layer, attached, manufactured onto or otherwise fixed to the second layer, should comprise a third hydrophilic or hydrophobic fibre layer such that a second size of a given grade of absorbent particles may pass into the spaces and or voids between the individual fibres that constitute the third layer but the same size of particles are unable to pass into the spaces or voids in the second described layer.

Any number of additional layers may be added to the structure depending upon the particle size distribution of the SAP being used within the end product under manufacture and the pre-determined distribution of SAP through the cross-section of the air-permeable, non-woven structure according to the absorbency performance required.

Step (ii) involves dispersing the absorbent particles onto the surface of the uppermost of the air-permeable non-woven structure by a controllable mechanical means such as precision scatter coating, whereby the particles are mechanically distributed onto the surface via a rotary screen. In scatter coating, the particles to be dispersed are usually conveyed to a rotary screen through a closed particle feeding tube by a worm drive device where the particles flow onto a doctor blade which pushes the particles through the holes in a purpose designed screen directly onto the surface of the target air-permeable structure. Other types of scatter coating mechanisms are also suitable.

An alternative method of dispersing the absorbent particles onto the uppermost surface of the structure is by powder spraying, whereby the absorbent particles are accelerated though an orifice, usually with the assistance of compressed air. By controlling the mechanical design of the orifice and the air pressure provided, it is possible accurately to meter a specific mass of particles through the orifice in a given period of time, thereby facilitating accurate dispersion of said particles and creating either pre-determined patterns of dispersion or full width dispersion of absorbent particles.

A further alternative method of dispersing the absorbent particles onto the uppermost surface of the structure is with the implementation of a vibrating particle feeder system. Vibrating feeders systems may be supplied with either electromagnetic or vibrator motor drives.

Electromagnetic drive feeders may be used where the delivery flow of particles requires frequent adjustment, or is subject to constant stop/start cycles, as would be the case where intermittent dispersion patterns are required on the surface of the substrate onto which the particles are to be dispersed.

Using one of aforementioned methods or any other method of controlled dispersion, the particles may be dispersed across the entire surface of the air permeable structure, or only across selected, pre-determined areas of the air permeable structure depending upon the design requirements of the end manufactured product.

Although the previously described methods of dispersing the particles onto the air permeable structure are relatively accurate, it is inevitable that some areas of the structure will initially have a higher areal density of particles on the surface of the air-permeable, non-woven structure than other areas due to the localised agglomeration of the particles and/or the motion of either the dispersal system or the motion of the air permeable structure onto which the particles have been dispersed. In-process airflow due to the motion of the air-permeable structure during the dispersal phase may also result in localised agglomeration of the dispersed particles.

Depending upon the specific particle size of the absorbent particles used and the specific type and construction of the uppermost layer of the air-permeable, non-woven structure, some of the particles will become entrained within the structure by falling through the uppermost layer at the surface as a result of gravity.

Other particles may come to rest partially entrained within the uppermost layer and partially or wholly above the next layer down and so on, depending upon the number of individual layers that make up the air permeable structure, the specific construction of those individual layers and the PSD of the particular grade of SAP being dispersed.

Preferably, the absorbent particles have a mean particle size of between >10 μm and <3000 μm. In preferred embodiments, the mean particle size of the absorbent particles is in the range of between 25 μm to 2500 μm, more preferably 50 μm to 1500 μm, and most preferably 75 μm to 1000 μm. In an alternative embodiment, the mean particle size of the absorbent particles is preferably in the range of between 40 μm to 900 μm.

A number of alternative technologies are suitable for dissipating the particles throughout the layered construction of the air permeable substrate in step (iii). These include but, are not limited to, the following:

A first alternative method of dissipating the dispersed particles is to subject the whole matrix of dispersed particles and air-permeable, non-woven structure to an externally applied vibration energy (VE) of a frequency between 1 Hz and 250 Hz, and with an amplitude of between 0.1 mm to 5 mm. In practice, such vibration energy is transmitted to the matrix via either an electromagnetic or vibrator motor drive connected to a plate system over which the matrix is transported whilst the vibration energy is applied.

The primary effect of subjecting the particles to the VE is to de-agglomerate the particles whilst at the same time agitating the particles so that the particles become located substantially below the level of the uppermost surface of the air-permeable structure onto which the particles were first dispersed. Each particle size will thus come to rest generally within a layer of the structure designed to accommodate that given particle size whilst preventing larger particles from passing through a given layer into any of the lower layers.

A second alternative method of dissipating the dispersed particles is to subject the matrix to an atmospheric pressure, alternating electrical field (AEF) of between 1 kV and 250 kV at a frequency of between 1 Hz to 250 Hz whilst the particles are substantially in intimate contact with the uppermost layer.

The primary effect of subjecting the particles to the AEF is to de-agglomerate the particles, whilst at the same time agitating the particles so that they become located substantially below the level of the uppermost surface of the structure onto which the particles were first dispersed. Each particle size will thus come to rest generally within a layer of the structure designed to accommodate that given particle size whilst preventing larger particles from passing through a given layer into any of the lower layers.

The effect of the combined pre-determined construction of the air-permeable, non-woven structure together with the specific grade and therefore PSD of the SAP selected will result in the particles becoming distributed throughout a cross-section of the air-permeable structure according to the specific size of a given amount of particles and the void space between the fibres that constitute each individual layer of the air permeable structure.

Simply described, the larger particle sizes will reside in the upper layers of the structure and the smaller particle sizes will reside in the lower layers of the structure but no particles will be able to pass through the entire structure.

Preferably, the frequency of the applied VE is in the range of 1 Hz to 250 Hz. In preferred embodiments, the frequency of the applied VE is in the range of 5 Hz to 200 Hz, preferably 10 Hz to 150 Hz, more preferably 20 Hz to 100 Hz, still more preferably 30 Hz to 80 Hz, further preferably 40 Hz-60 Hz and most preferably 45 Hz-55 Hz.

Preferably, the amplitude of the applied VE is in the range of 0.1 mm to 5 mm. In preferred embodiments, the amplitude of the applied VE is 0.2 mm to 4 mm, preferably 0.3 mm to 3 mm, more preferably 0.4 mm to 2 mm and most preferably 0.5 mm to 1.5 mm.

Preferably, the voltage applied to generate the AEF is in the range of 1 kV to 1000 kV. In preferred embodiments the voltage applied to generate the AEF is in the range of 10 kV to 1000 kV, preferably 20 kV to 500 kV, more preferably 30 kV to 200 kV, still more preferably 40 kV to 100 kV and most preferably 50 kV to 75 kV. In an alternative embodiment, the voltage applied to generate the AEF is preferably in the range of 10 kV to 50 kV.

Preferably, the power supply of the AEF is in the frequency range of between 1 Hz-100 kHz. In preferred embodiments the power supply of the AEF is in the frequency range of between 1 Hz to 250 Hz, more preferably 10 Hz to 100 Hz, still more preferably 20 Hz-60 Hz, and most preferably between 50 Hz to 60 Hz.

The AEF may be configured with sinusoidal or square-wave alternating currents between utility frequencies of 50 Hz to 60 Hz or with pulsed wave forms depending upon the variable conditions of the absorbent particle size, the rate of dispersion of the particles onto the surface of the air-permeable non-woven structure and the construction, density and thickness of the structure.

A third alternative method of dissipating the dispersed particles is to subject the matrix to a high frequency vibration source such as that provided by ultra-sonic vibrating sonotrode, whilst the particles are substantially in intimate contact with the uppermost layer onto which the particles have been dispersed.

A fourth alternative method of dissipating the dispersed particles is to subject the matrix to a vacuum process, whilst the particles are substantially in intimate contact with the uppermost layer onto which the particles have been dispersed, the vacuum being applied from beneath the lowermost layer to draw the dispersed particles into the entire layered structure of the matrix.

An optional fourth step is to consolidate the particles within the layers of the air-permeable substrate, if required. In some cases, it will not be required or desired, due to the specific construction of the end product, to consolidate the particles within the air permeable structure. For example, if the SAP is dispersed and then dissipated throughout the air-permeable structure in line with the end product manufacturing process of an infant diaper then a next step of the production process would be to attach a polymer film to the uppermost layer of the structure. This polymer sheet is often referred to within the diaper manufacturing industry as the back sheet.

If however, the SAP dispersed and dissipated air permeable structures are to be incorporated into the end product in a second manufacturing process, then it may become necessary to consolidate the SAP particles within the air-permeable structure so that the particles do not become dislodged during transport to a second production site or during the secondary end product manufacturing phase.

There are many ways of consolidating the particles within air-permeable structure layers depending upon the specific construction and the specific materials that make up the air-permeable structure and the type and size of absorbent particles that are dispersed and dissipated within the matrix.

One example of a method of consolidating the SAP within the air permeable structure is to construct the uppermost layer of the structure to include fibres that can be made to extend and shrink at differential rates in length when subjected to an external heating source. Said fibres thus crimp and lock adjacent fibres together, having the effect of reducing the spaces and or voids between the fibres thereby to prohibit previously dissipated particles from exiting the air-permeable structure, whilst retaining the ability of the matrix to acquire, distribute and absorb liquid waste in an efficient manner.

A second example of a method of consolidating the SAP within the air-permeable structure is to attach a further layer of non-woven fabric to the surface of the uppermost layer of the structure after the entire matrix has been subjected to a dissipation process, thereby mechanically prohibiting the now entrained SAP from exiting the air permeable structure. The further layer of non-woven fabric may have hydrophobic or hydrophilic properties and may be attached to the uppermost layer by welding, gluing, bonding, stitching or other suitable means compatible with the desired properties of the end product to be manufactured.

A third example of a method of consolidating the SAP within the air-permeable structure is to incorporate lower melt temperature fibres together with higher melt temperature fibres within the uppermost layer. After the entire matrix has been subjected to a dissipation process, an external heat source may be applied to the uppermost layer of the structure so that the lower melt temperature fibres soften and become tacky thereby adhering to the surface of the SAP particles located in the uppermost layer. Upon cooling, the SAP in the uppermost layer will become bonded to the fibres that form the layer, and create a physical barrier to those smaller particles already entrained within the lower levels of the structure and prevent the egress of said particles from within the air-permeable structure.

A fourth example of a method of consolidating the SAP within the air permeable structure is to incorporate a binding agent or agents within the air-permeable structure during the manufacture of the structure itself. After the SAP has been dispersed on the uppermost surface and been subjected to an AEF to de-agglomerate and dissipate the, the entire matrix is subjected to an external heat source to activate the binder or binders within the air-permeable structure, thereby causing the binder or binders to lock the SAP within the structure.

In some circumstances, it may also be desirable to ensure that no dissipated SAP within the air-permeable structure can egress the structure in post-dissipation processes such as slitting.

The method of the present invention may also include further steps of cutting the air-permeable non-woven structure, and subsequently sealing the edges thereof. Suitable methods for such sealing include, but are not limited to: ultra-sonic welding, radio frequency welding, heat welding, impulse welding, stitching, binding, bonding and other similar processes.

In order that the present invention may be fully understood, preferred embodiments thereof will now be described in detail, though only by way of example, with reference to the accompanying drawings in which:

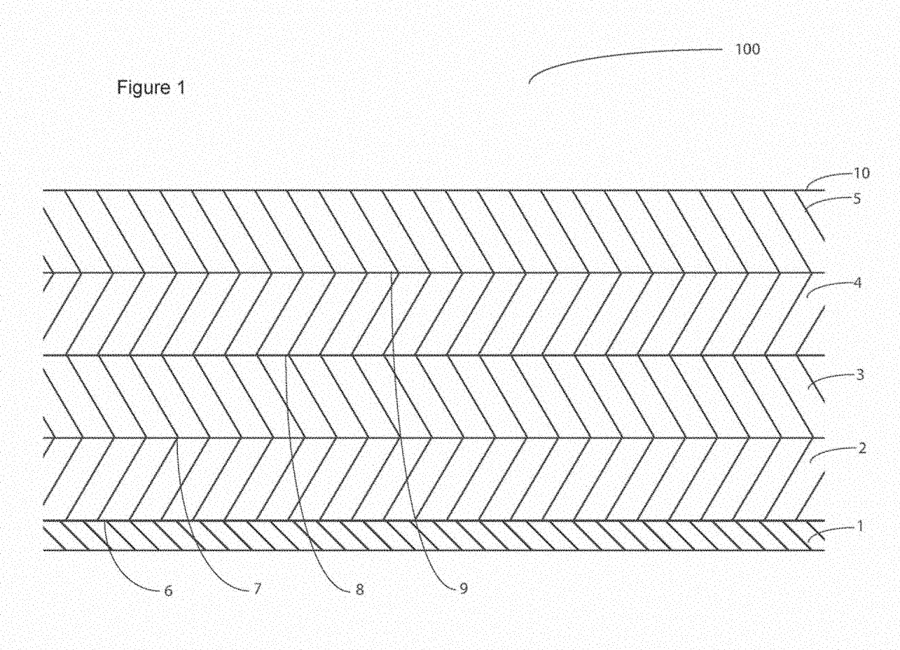

FIG. 1 is a cross-sectional view of an air-permeable, non-woven structure as formed in step (i) of the method of the present invention;

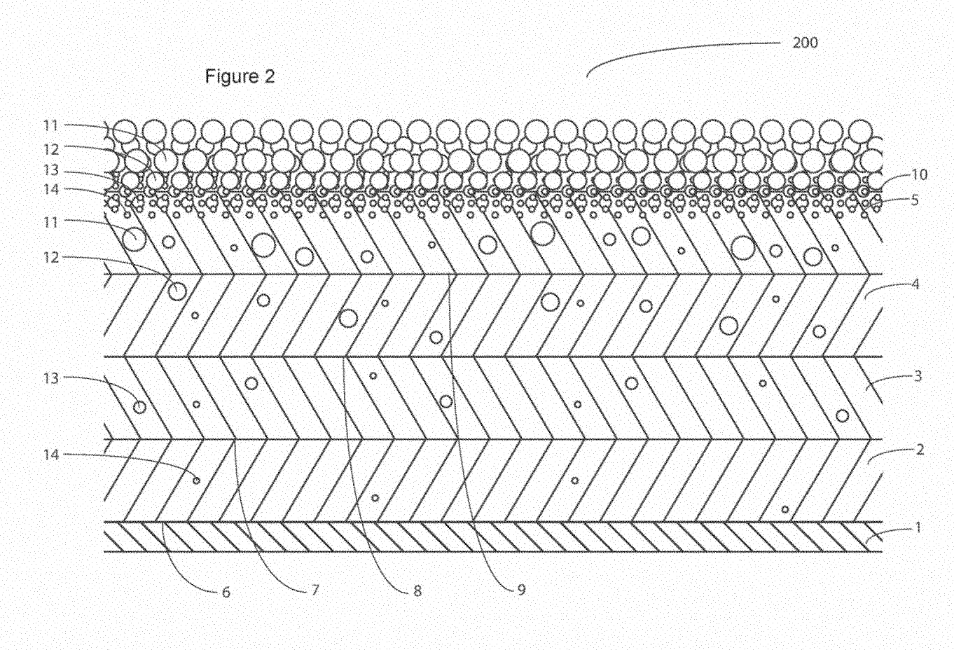

FIG. 2 is a cross-sectional view of the air-permeable, non-woven structure of FIG. 1, having absorbent particles dispersed thereon, as per step (ii) of the method of the present invention;

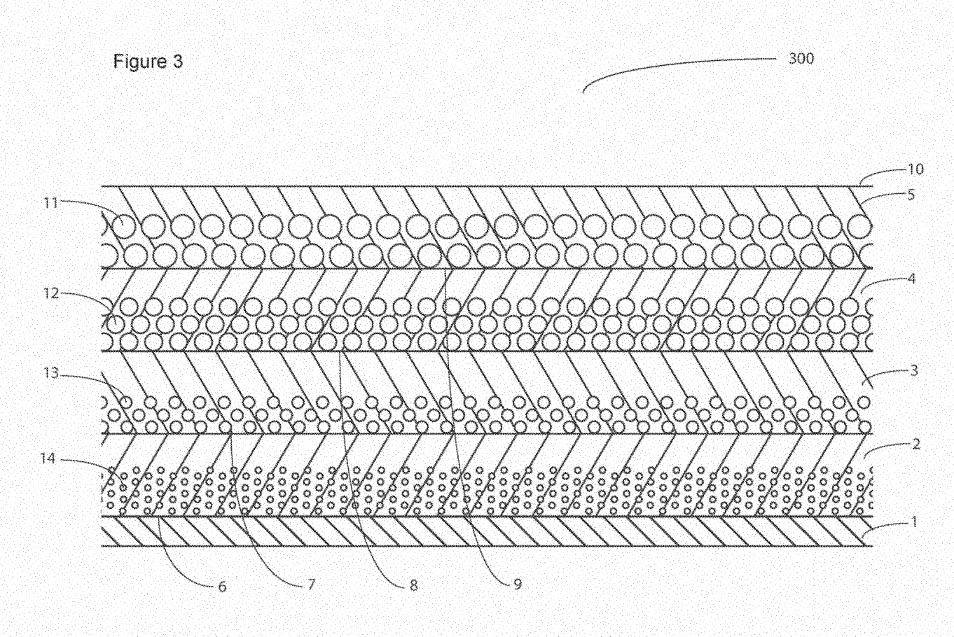

FIG. 3 is a cross-sectional view of the air-permeable, non-woven structure of FIG. 2, following dissipation of the absorbent particles, as per step (iii) of the method of the present invention;

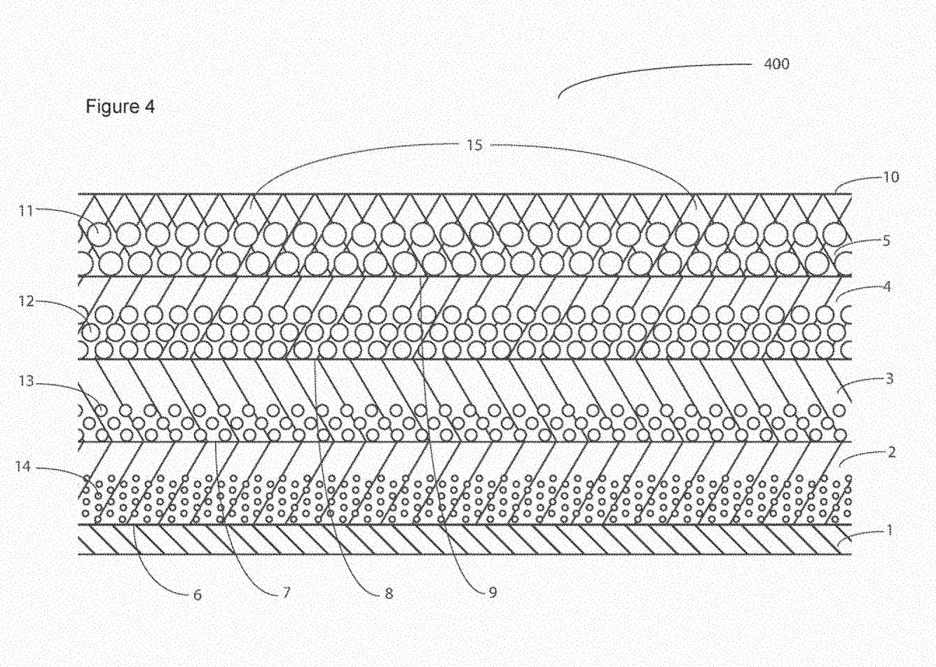

FIG. 4 is a cross-sectional view of the air-permeable, non-woven structure of FIG. 3, following consolidation of the absorbent particle and non-woven matrix, as per step (iv) of the method of the present invention;

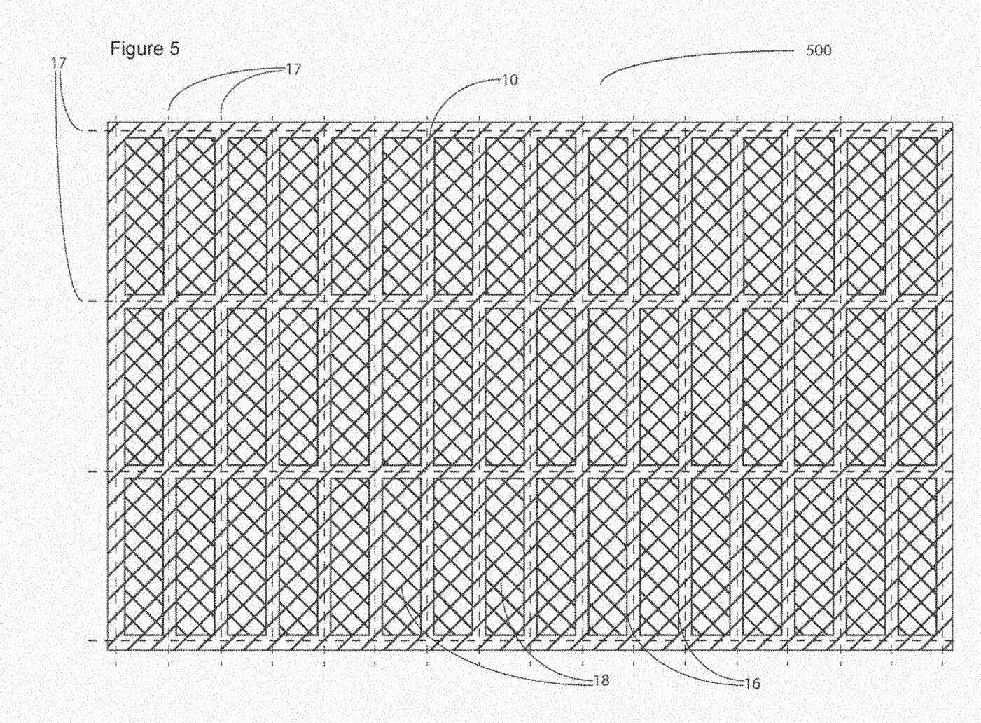

FIG. 5 is a top view of an absorbent particle and non-woven matrix having absorbent particles arranged in a pre-determined pattern;

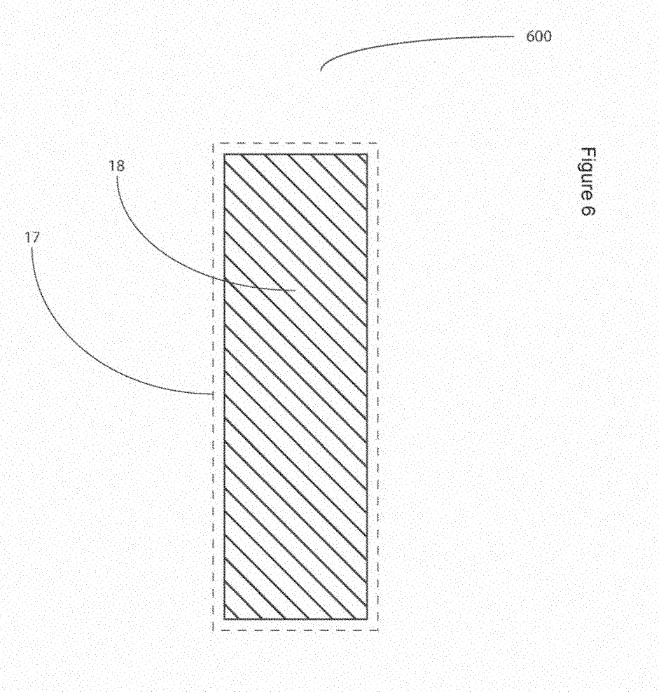

FIG. 6 is a top view of an absorbent article, having been cut from the matrix of FIG. 5;

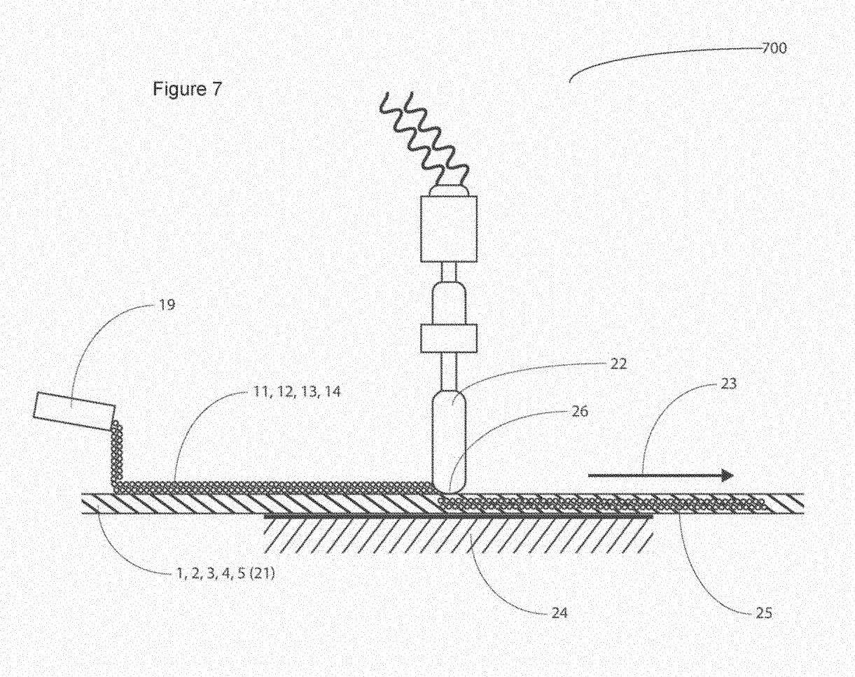

FIG. 7 is an illustration of a process layout for performing steps (ii) and (iii) of a first embodiment of the method of the present invention;

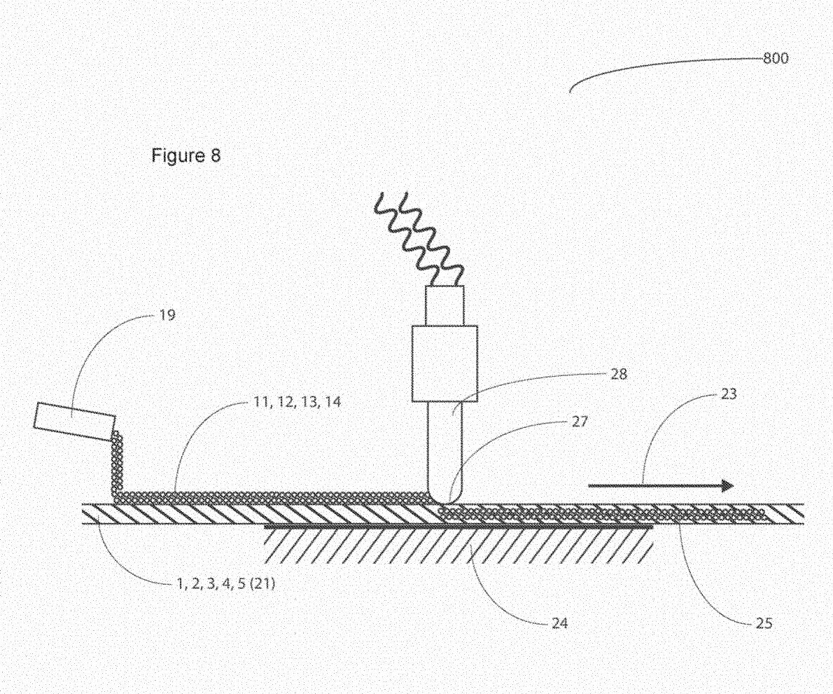

FIG. 8 is an illustration of a process layout for performing steps (ii) and (iii) of a second embodiment of the method of the present invention;

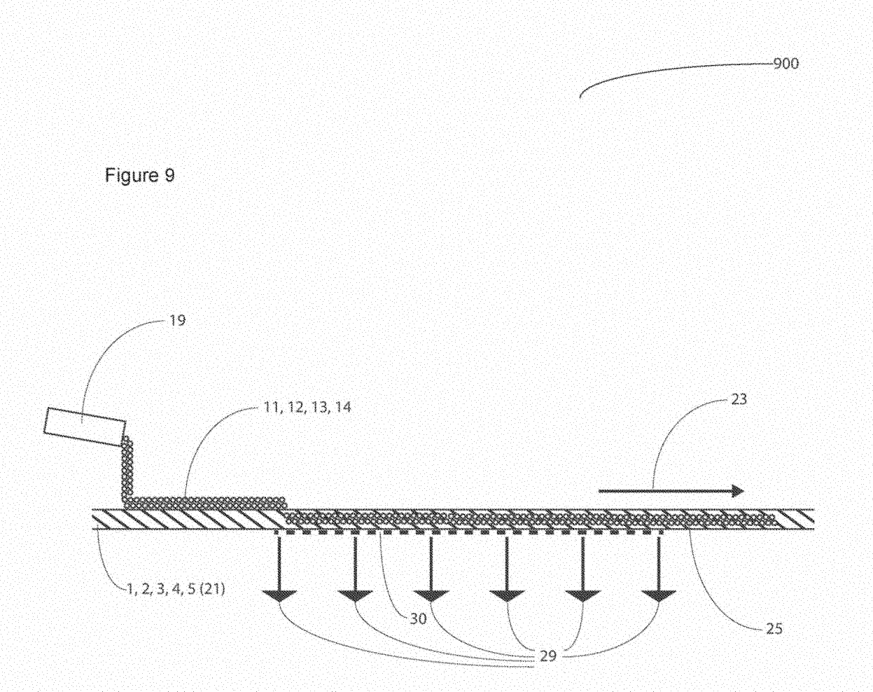

FIG. 9 is an illustration of a process layout for performing steps (ii) and (iii) of a third embodiment of the method of the present invention; and

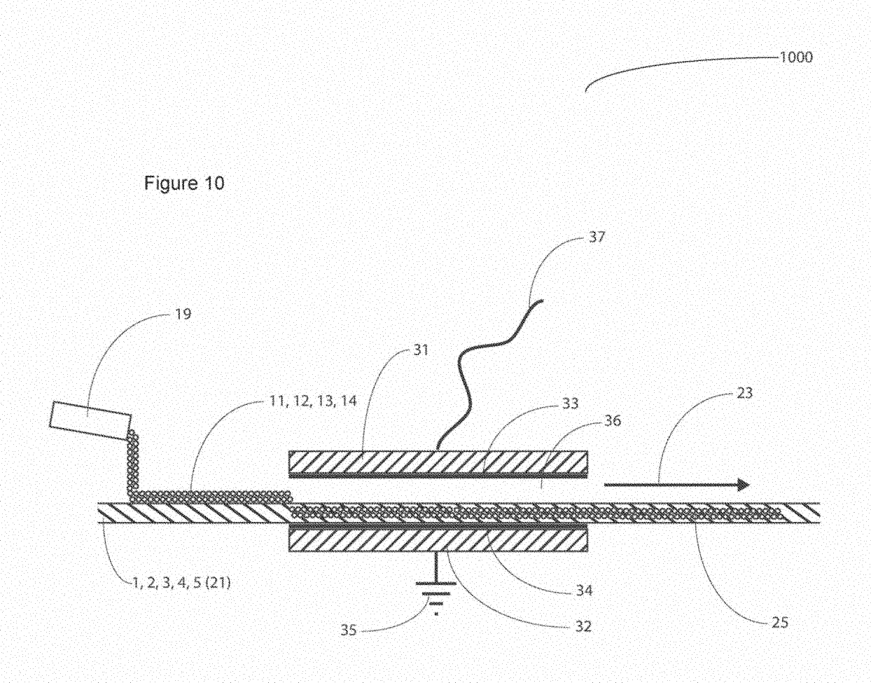

FIG. 10 is an illustration of a process layout for performing steps (ii) and (iii) of a fourth embodiment of the method of the present invention.

Referring first to FIG. 1, there is shown an air-permeable, non-woven structure, generally indicated 100, as formed in step (i) of the method of the present invention. A first layer 1 comprised of non-woven fibres is bonded, manufactured onto or otherwise attached to a second layer 2 at interface 6; a third layer 3 is bonded, manufactured onto or otherwise attached to the second layer 2 at interface 7; a fourth layer 4 is bonded, manufactured onto or otherwise attached to the third layer 3 at interface 8; and a fifth layer 5 is bonded, manufactured onto or otherwise attached to the fourth layer 4 at interface 9. The surface 10 of the fifth layer 5 in this embodiment is the uppermost surface. The fifth layer 5 comprises bi-component fibres in which the two components have differential melting temperatures.

Referring now to FIG. 2, there is shown the air-permeable, non-woven structure described above with reference to FIG. 1, now generally indicated 200. Absorbent particles 11, 12, 13 and 14 of varying particle sizes, have been dispersed onto the uppermost surface 10 of the fifth layer 5, as per step (ii) of the method of the present invention. Due to the relatively open structure of the surface of the fifth layer 5, some of the particles 11, 12, 13 and 14 have passed into the subsequent layers 2, 3, 4 and 5. Some of the absorbent particles have thus come to rest in layers where the void space within that specific layer allows free passage of the absorbent particles. Other particles 11, 12, 13 and 14 have come to rest partially above and partially below the surface of the fifth layer 5 such that absorbent particles 11 are prevented from passing into the fourth layer 4 at interface 9 due to the restrictive spaces between the fibres that constitute the fourth layer 4; whilst absorbent particles 12 are prevented from passing into the third layer 3 at interface 8 due to the restrictive spaces between the fibres that constitute the third layer 3; absorbent particles 13 are prevented from passing into the second layer 2 at interface 7 due to the restrictive spaces between the fibres that constitute the second layer 2; whilst the restrictive void spaces between the fibres that constitute the first layer 1 are too small to allow the passage of any of the absorbent particles, irrespective of their size.

Referring now to FIG. 3, there is shown the air-permeable, non-woven structure described above with reference to FIGS. 1 and 2, now generally indicated 300. The structure 300, and the dispersed absorbent particles 11, 12, 13 and 14 have now been exposed to a particle dissipation process as per step (iii) of the method of the present invention. The absorbent particles 11, 12, 13 and 14 have been de-agglomerated and as a result of the effect of the dissipation process have become located substantially below the uppermost surface 10 of the fifth layer 5 and have become substantially dispersed throughout the structure according to both the void space size within each of the individual layers 2, 3, 4 and 5 and the specific particle size distribution profile of the absorbent being dispersed. Particles 11, 12, 13 and 14 have come to rest substantially below the surface of the fifth layer 5; absorbent particles 11 are prevented from passing into the fourth layer 4 at interface 9 due to the restrictive spaces between the fibres that constitute the fourth layer 4; absorbent particles 12 are prevented from passing into the third layer 3 at interface 8 due to the restrictive spaces between the fibres that constitute the third layer 3; absorbent particles 13 are prevented from passing into the second layer 2 at interface 7 due to the restrictive spaces between the fibres that constitute the second layer 2; whilst the restrictive void spaces between the fibres that constitute layer 1 are too small to allow the passage of any of the absorbent particles, irrespective of their size.

Referring now to FIG. 4, there is shown the air-permeable, non-woven structure described above with reference to FIGS. 1 to 3, now generally indicated 400, following the performance of a consolidation process, as per optional step (iv) of the method of the present invention. The consolidation process has been applied to the uppermost (fifth) layer 5 such that now dissipated particles 11, 12, 13 and 14 are prevented from exiting the air-permeable structure via the uppermost (fifth) layer 5 due to the consolidation of fibres in region 15 of the fifth layer 5.

Referring now to FIG. 5, there is shown an absorbent particle and non-woven matrix, generally indicated 500, emerging from method step (iv), as described above with reference to FIG. 4. In this embodiment, the uppermost surface 10 of the fifth layer 5 has had absorbent particles dispersed upon it in a pre-determined pattern or shape 16. Following exposure to a suitable dissipation process (iii) followed by consolidation (iv) of the particles, an portion 18 of the matrix 500 may be cut or otherwise extracted from the air permeable non-woven matrix 500 along path 17, such that the path of the cut line 17 is larger in size than the pre-determined pattern or shape 16.

Referring now to FIG. 6, there is shown an absorbent article generally indicated 600, following cutting or otherwise extracting the portion 18 of the particle and non-woven matrix 500 along path 17, as described above with reference to FIG. 5.

Referring now to FIG. 7, there is shown a process layout 700 for performing steps (ii) and (iii) of a first embodiment of the method of the present invention. Absorbent particles 11, 12, 13 and 14 are dispersed, as per step (ii), onto the surface of an air-permeable, non-woven structure 21, comprising layers 1, 2, 3, 4 and 5, by means of a controlled scattering device 19. The non-woven structure 21 is moved in the direction of arrow 23 across a base plate 24 whilst simultaneously a substantially vertically applied ultra-sonic force is applied, as per step (iii), at the interface 26 of an ultra-sonic device 22 causing the particles 11, 12, 13 and 14 to become entrained within the non-woven layers 25.

Referring now to FIG. 8, there is shown a process layout 800 for performing steps (ii) and (iii) of a second embodiment of the method of the present invention. Absorbent particles 11, 12, 13 and 14 are dispersed, as per step (ii) onto the surface of an air-permeable, non-woven structure 21, comprising layers 1, 2, 3, 4 and 5 by means of a controlled scattering device 19. The non-woven layer 21 is moved in the direction of arrow 23 across a base plate 24 whilst simultaneously a substantially vertically applied low frequency vibration in the range of 10 Hz to 200 Hz is applied, as per step (iii), at the interface 27 of a low frequency vibration device 28 causing the particles 11, 12, 13 and 14 to become entrained within the non-woven layers 25.

Referring now to FIG. 9, there is shown a process layout 900 for performing steps (ii) and (iii) of a third embodiment of the method of the present invention. Absorbent particles 11, 12, 13 and 14 are dispersed, as per step (ii), onto the surface of an air-permeable, non-woven structure 21, comprising layers 1, 2, 3, 4 and 5, by means of a controlled scattering device 19. The non-woven structure 21 is moved in the direction of arrow 23 across a perforated plate or membrane 30 whilst simultaneously a substantially vertically applied vacuum force 29 is applied, as per step (iii), from beneath the perforated plate or membrane 30 causing the particles 11, 12, 13 and 14 to become entrained within the non-woven layers 25.

Referring finally to FIG. 10, there is shown a process layout 1000 for performing steps (ii) and (iii) of a fourth embodiment of the method of the present invention. Absorbent particles 11, 12, 13 and 14 are dispersed, as per step (ii) onto the surface of an air-permeable, non-woven an air-permeable, layers 1, 2, 3, 4 and 5, by means of a controlled scattering device 19. The non-woven structure 21 is moved in the direction of arrow 23 between an upper electrode device 31 and a lower electrode device 32, each electrode device having a dielectric plate 33 and 34 located between said electrode devices 31 and 32 and the target particles 11, 12, 13 and 14 together with the non-woven structure 21. The upper electrode device 31 is connected to a high voltage, alternating current generator via cable 37 whilst the lower electrode device 32 is connected to earth 35. As the dispersed powder 11, 12, 13 and 14 is transported into the zone 36 between the two dielectric plates 33 and 34, the energy field generated between the upper electrode 31 and lower electrode 32 causes the powder to become excited and vibrate in a plane substantially normal to the plane of the dielectric plates 33 and 34 and become dissipated, as per step (iii), within the non-woven layers 25.

The method according to the present invention will now be further described by way of the following examples:

### Example 1

A non-woven fabric structure was manufactured comprising five individual layers of varying void space dimensions between individual and groups of fibres. Each of the four lower layers were manufactured using a blend of polypropylene, polyethylene and polyester fibres in the ratio of 20%, 40% and 40% respectively.

The construction of the entire non-woven structure was such that the lowermost layer or first layer (ADL) would not permit the entry into or passage through of any Super Absorbent Polymer (SAP) particles of less than 10 μm in minimum diameter. Effectively, this layer was manufactured to act as a physical barrier to the passage of any of the SAP to be processed within this entire example.

The next layer or second layer was manufactured to permit the entry of SAP particles in the range of 10 μm to 150 μm but not to allow the entry of or passage through of SAP particles of particle size greater than 150 μm.

The next or third layer was manufactured to permit the entry of SAP particles in the range 10 μm to 400 μm but not to permit the entry of or passage through of SAP of particle size greater than 400 μm.

The next or fourth layer was manufactured to permit the entry of SAP particles in the range 10 μm to 600 μm but not to permit the entry or passage through of SAP of particle size greater than 600 μm.

The next or fifth layer was manufactured to permit the entry of SAP particles in the range 10 μm and above. The uppermost layer of the non-woven structure was manufactured from bi-component fibres in a side by side configuration from polyester and polyethylene.

The method of manufacture of the non-woven substrate as a whole was a combination of conventional random carding, air through and needle punch processes.

A sodium polyacrylate SAP of particle size distribution ranging between 40 μm to 850 μm commercially manufactured by a market leader in the hygiene materials supply market was mechanically dispersed onto the uppermost surface of the aforesaid high loft non-woven fabric at an areal dispersion rate of 325 gsm (gm⁻²) which in practice would equate to an amount of 13 g in a typical infant diaper core.

The SAP and non-woven fabric structure was then subjected to an external vibrating energy source of frequency 50 Hz and amplitude 1.5 mm to dissipate the SAP particles within the structure of the non-woven. The particles were caused to come to rest within the structure in a gradient manner according to the specific PSD of the SAP particles and the void space at a given location within the non-woven fabric structure.

Following the dissipation process, the uppermost layer of the non-woven fabric structure was then subjected to an external heat source provided by infra-red heating lamps, to cause the bi-component fibres in the uppermost layer of the structure to crimp as a result of differential expansion of each component of the bi-component fibres.

The entire non-woven fabric structure and SAP matrix was then allowed to cool. After cooling it was found that the SAP was fully contained within the non-woven structure.

### Example 2

A non-woven fabric structure was manufactured comprising five individual layers of varying void space dimensions between individual and groups of fibres. Each of the four lower layers were manufactured using a blend of polypropylene, polyethylene and polyester fibres in the ratio of 20%, 40% and 40% respectively.

The construction of the non-woven structure was such that the lowermost layer or first layer (ADL) would not permit the entry into or passage through of any Super Absorbent Polymer particles (SAP) of less than 10 μm in minimum diameter. Effectively, this layer was manufactured to act as a physical barrier to the passage of any of the SAP to be processed within this example.

The next layer or second layer was manufactured to permit the entry of SAP particles in the range of 10 μm to 150 μm but not to allow the entry of or passage through of SAP of particle size greater than 150 μm.

The next or third layer was manufactured to permit the entry of SAP particles in the range 10 μm to 400 μm but not to permit the entry of or passage through of SAP of particle size greater than 400 μm.

The next or fourth layer was manufactured to permit the entry of SAP particles in the range 10 μm to 600 μm but not to permit the entry or passage through of SAP of particle size greater than 600 μm.

The next or fifth layer was manufactured to permit the entry of SAP particles in the range 10 μm and above. The uppermost layer of the non-woven structure was manufactured from bi-component fibres in a side by side configuration from polyester and polyethylene.

The method of manufacture of the non-woven substrate as a whole was a combination of conventional random carding, air through and needle punch processes.

A sodium polyacrylate SAP of particle size distribution ranging between 40 μm to 850 μm commercially manufactured by a market leader in the hygiene materials supply market was mechanically dispersed onto the uppermost surface of the aforesaid high loft non-woven fabric at an areal dispersion rate of 325 gsm (gm⁻²) which in practice would equate to an amount of 13 g in a typical infant diaper core.

The SAP and non-woven fabric structure was then subjected to an alternating voltage energy field (AVEF) of 25 kV and of frequency 50 Hz between two opposed electrode plates placed 10 mm apart along their longitudinal axis to excite the SAP particles such that the particles were made to vibrate in a direction normal to the opposed surfaces of the electrode plates, the vibration energy being sufficient to dissipate the SAP within the non-woven fabric structure such that no SAP remained on the uppermost surface of the non-woven fabric structure.

Following the dissipation process the entire non-woven structure and SAP matrix was subject to an air-through heating process such that the lower melting temperature component of the bi-component fibres that constitute the uppermost layer of the structure became soft and tacky such as to bond to adjacent individual and groups of fibres thereby entrapping the SAP dissipated within the uppermost layer and creating a physical barrier to those SAP particles dissipated within the adjacent and lower layer from passing out through the uppermost layer upon the entire matrix being subjected to agitation.

Upon cooling, the lower melting temperature component of the bi-component fibres in the uppermost layer of the structure retained their solid state now bonded to adjacent individual and groups of fibres.

### Example 3

A non-woven fabric structure was manufactured comprising five individual layers of varying void space dimensions between individual and groups of fibres. Each of the four lower layers were manufactured using a blend of polypropylene, polyethylene and polyester fibres in the ratio of 20%, 40% and 40% respectively.

The construction of the entire non-woven structure was such that the lower most layer or first layer (ADL) would not permit the entry into or passage through of any Super Absorbent Polymer particles (SAP) of less than 10 μm in minimum diameter. Effectively, this layer was manufactured to act as a physical barrier to the passage of any of the SAP to be processed within this example.

The next layer or second layer was manufactured to permit the entry into of SAP particles in the range of 10 μm to 150 μm but not to allow the entry of or passage through of SAP of particle size greater than 150 μm.

The next or third layer was manufactured to permit the entry of SAP particles in the range 10 μm to 400 μm but not to permit the entry of or passage through of SAP of particle size greater than 400 μm.

The next or fourth layer was manufactured to permit the entry of SAP particles in the range 10 μm to 600 μm but not to permit the entry or passage through of SAP of particle size greater than 600 μm.

The next or fifth layer was manufactured to permit the entry of SAP particles in the range 10 μm and above. The uppermost layer of the non-woven structure was manufactured from bi-component fibres in a side by side configuration from polyester and polyethylene.

The method of manufacture of the non-woven substrate as a whole was a combination of conventional random carding, air through and needle punch processes.

A sodium polyacrylate SAP of particle size distribution ranging between 40 μm to 850 μm commercially manufactured by a market leader in the hygiene materials supply market was mechanically dispersed onto the uppermost surface of the aforesaid high loft non-woven fabric structure at an areal dispersion rate of 325 gsm (gm⁻²) which in practice would equate to an amount of 13 g in a typical infant diaper core.

The SAP and non-woven fabric structure was then subjected to a vacuum, applied from below the lowermost surface of the structure of greater than 1 bar in pressure such that the SAP particles dispersed upon the uppermost layer of the structure were drawn into the structure with the SAP particles coming to rest within a specific given layer dependent upon the given diameter of the SAP particle in question.

Following the dissipation process as a result of the applied vacuum, a sixth layer of spun bonded polypropylene non-woven fabric of areal weight of 8 gsm (gm⁻²) was then attached to the uppermost surface of the SAP and non-woven fabric matrix by means of intermittent ultra-sonic welding such that the SAP particles now dissipated within the non-woven fabric structure were prohibited from egress via the uppermost layer of the structure.

### Example 4

A non-woven fabric structure was manufactured comprising five individual layers of varying void space dimensions between individual and groups of fibres. Each of the four lower layers were manufactured using a blend of polypropylene, polyethylene and polyester fibres in the ratio of 20%, 40% and 40% respectively.

The construction of the entire non-woven structure was such that the lower most layer or first layer (ADL) would not permit the entry into or passage through of any Super Absorbent Polymer particles (SAP) of less than 10 μm in minimum diameter. Effectively, this layer was manufactured to act as a physical barrier to the passage of any of the SAP to be processed within this example.

The next layer or second layer was manufactured to permit the entry into of SAP particles in the range of 10 μm to 150 μm but would not allow the entry of or passage through of SAP of particle size greater than 150 μm.

The next or third layer was manufactured to permit the entry of SAP particles in the range 10 μm to 400 μm but would not permit the entry of or passage through of SAP of particle size greater than 400 μm.

The next or fourth layer was manufactured to permit the entry of SAP particles in the range 10 μm to 600 μm but would not permit the entry or passage through of SAP of particle size greater than 600 μm.

The next or fifth layer was manufactured to permit the entry of SAP particles in the range 10 μm and above. The uppermost layer of the non-woven structure was manufactured from bi-component fibres in a side by side configuration from polyester and polyethylene.

The method of manufacture of the non-woven substrate as a whole was a combination of conventional random carding, air through and needle punch processes.

A sodium polyacrylate SAP of particle size distribution ranging between 40 μm to 850 μm commercially manufactured by a market leader in the hygiene materials supply market was mechanically dispersed onto the uppermost surface of the aforesaid high loft non-woven fabric structure at an areal dispersion rate of 325 gsm (gm⁻²) which in practice would equate to an amount of 13 g in a typical infant diaper core.

The SAP and non-woven fabric structure was then subjected to an external vibrating energy source of frequency 50 Hz and amplitude 1.5 mm to dissipate the SAP particles within the structure of the non-woven. The particles were caused to come to rest within the substrate in a gradient manner according to the specific PSD of the SAP particles and the void space at a given location within the cross-section of the entire non-woven fabric structure.

Following the dissipation process, a polyethylene film, coated on one surface with a heat sensitive adhesive based on polyurethane chemistry, was bonded onto the uppermost surface of the uppermost layer such that the SAP particles now dissipated within the non-woven fabric structure were prohibited from egress via the uppermost layer of the substrate.

### Example 5

A non-woven fabric structure was manufactured comprising five individual layers of varying void space dimensions between individual and groups of fibres. Each of the four lower layers were manufactured using a blend of polypropylene, polyethylene and polyester fibres in the ratio of 20%, 40% and 40% respectively.

The construction of the entire non-woven structure was such that the lowermost layer or first layer (ADL) would not permit the entry into or passage through of any Super Absorbent Polymer particles (SAP) of less than 10 μm in minimum diameter. Effectively, this layer was manufactured to act as a physical barrier to the passage of any of the SAP to be processed within this example.

The next layer or second layer was manufactured to permit the entry into of SAP particles in the range of 10 μm to 150 μm but not to allow the entry of or passage through of SAP of particle size greater than 150 μm.

The next or third layer was manufactured to permit the entry of SAP particles in the range 10 μm to 400 μm but not to permit the entry of or passage through of SAP of particle size greater than 400 μm.

The next or fourth layer was manufactured to permit the entry of SAP particles in the range 10 μm to 600 μm but not to permit the entry or passage through of SAP of particle size greater than 600 μm.

The next or fifth layer was manufactured to permit the entry of SAP particles in the range 10 μm and above. The uppermost layer of the non-woven structure was manufactured from bi-component fibres in a side by side configuration from polyester and polyethylene.

The method of manufacture of the non-woven substrate as a whole was a combination of conventional random carding, air through and needle punch processes. A sodium polyacrylate Super Absorbent Polymer (SAP) of particle size distribution ranging between 40 μm to 850 μm commercially manufactured by a market leader in the hygiene materials supply market was mechanically dispersed onto the uppermost surface of the aforesaid high loft non-woven fabric at an areal dispersion rate of 325 gsm (gm⁻²) which in practice would equate to an amount of 13 g in a typical infant diaper core.

The SAP, now dispersed onto the uppermost surface of the non-woven fabric, was then subjected to an ultra-sonic energy source of 15 kHz and with an amplitude of 90 microns via a suitably engineered sonotrode to excite the SAP particles such that the particles were made to vibrate in a direction normal to surface of the non-woven fabric structure, the vibration energy being sufficient to dissipate the SAP within the non-woven fabric structure such that no SAP remained on the uppermost surface of the non-woven fabric structure.

Following the dissipation process the non-woven structure and SAP matrix was subject to an external heating process such that the lower melting temperature component of the bi-component fibres that constitute the uppermost layer of the structure became soft and tacky such as to bond to adjacent individual and groups of fibres thereby entrapping the SAP dissipated within the uppermost layer and creating a physical barrier to those SAP particles dissipated within the adjacent and lower layers from passing out through the uppermost layer upon the entire matrix being subjected to agitation.

Upon cooling, the lower melting temperature component of the bi-component fibres in the uppermost layer of the structure retained its solid state now bonded to adjacent individual and groups of fibres.

1. A method for dissipating and entrapping absorbent particles within air permeable structures, for use in the construction of absorbent articles, said method comprising the steps of:
constructing an air-permeable, non-woven structure comprising at least first, second and third layers of non-woven fabric, each said numbered layer (n) being bonded, manufactured onto or otherwise joined to each subsequently numbered layer (n+1), and wherein fibres in each said layer are arranged so as to define void spaces therebetween of pre-determined size, corresponding to a given absorbent particle size distribution range;
and wherein the void spaces in each said numbered layer (n), are of smaller size than the void spaces in each subsequently numbered layer (n+1);
(ii) dispersing, by controllable mechanical means, absorbent particles onto an external surface of the highest numbered layer of said air-permeable, non-woven structure formed in step (i), said absorbent particles having a pre-determined particle size distribution range;
(iii) dissipating said dispersed absorbent particles within the air-permeable, non-woven structure by applying an external energy source acting upon the absorbent particles in a direction substantially normal to the plane of the external surface of the air-permeable, non-woven structure.

2. A method as claimed in claim 1, wherein the external energy source in step (iii) is a low frequency vibration source generating a frequency in the range of between 10 Hz and 200 Hz, and an amplitude in the range of between 0.1 mm and 5 mm.

3. A method as claimed in claim 1, wherein the external energy source in step (iii) is an alternating electric field generating a frequency in the range of between 10 Hz and 200 Hz and a voltage in the range of between 5 kV and 50 kV.

4. A method as claimed in claim 1, wherein the external energy source in step (iii) generates a vacuum pressure of at least 1×10⁵ Nm⁻² applied from below the first said layer of the air-permeable, non-woven structure.

5. A method as claimed in claim 1, wherein the external energy source in step (iii) is an ultra-sonic vibration source generating a frequency in the range of between 10 kHz and 50 kHz, and an amplitude in the range of between 5 microns (μm) and 500 microns (μm).

6. A method as claimed in any of the preceding claims wherein the highest numbered layer of the air-permeable, non-woven structure comprises bi-component fibres, each said component having differing thermal expansion properties.

7. A method as claimed in any of the preceding claims wherein each layer of the air-permeable, non-woven structure, with the exception of the first said layer, comprises bi-component fibres, each said component having differing thermal expansion properties.

8. A method as claimed in any of the preceding claims, wherein at least one of the layers of the air-permeable, non-woven structure comprises a blend of at least two different fibre types having differing thermal expansion properties.

9. A method as claimed in any of claims 6 to 8, further comprising the step, after the dissipation step (iii), of:
(iv) subjecting at least one layer of the air-permeable, non-woven structure to an external heat source, and subsequently effecting or allowing cooling of said at least one layer, so as to entrap said absorbent particles within said air-permeable, non-woven structure.

10. A method as claimed in claim 9 when dependent upon claim 6, wherein in step (iv), the highest numbered layer of the air-permeable, non-woven structure is subjected to an external heat source, such that said components in said bi-component fibres expand upon heating and contract upon cooling at differential rates, causing said fibres to curl or crimp, thereby entrapping said absorbent particles within said highest numbered layer.

11. A method as claimed in claim 9 when dependent upon claim 7, wherein in step (iv), all of the layers of the air-permeable, non-woven structure are subjected to an external heat source, such that said components in said bi-component fibres expand upon heating and contract upon cooling at differential rates, causing the fibres to curl or crimp, thereby entrapping said absorbent particles within said air-permeable, non-woven structure.

12. A method as claimed in claim 9 when dependent upon claim 8, wherein in step (iv), all of the layers of the air-permeable, non-woven structure are subjected to an external heat source, such that the fibres having the lowest melting temperature soften and become tacky so as to adhere to adjacent absorbent particles thereby entrapping said absorbent particles within said air-permeable, non-woven structure.

13. A method as claimed in any of the preceding claims, further comprising the step of:
(v) welding, bonding or otherwise attaching a further layer to the external surface of the highest numbered layer of said air-permeable, non-woven structure incorporating said dissipated absorbent particles.

14. A method as claimed in claim 13, wherein said further layer is a layer of non-woven fabric.

15. A method as claimed in claim 13, wherein said further layer is a polymer film.

16. A method as claimed in any of the preceding claims wherein the absorbent particles are organic.

17. A method as claimed in any of claims 1 to 15, wherein the absorbent particles comprise sodium polyacrylate or a polymer blend incorporating sodium polyacrylate.

18. A method as claimed in any of the preceding claims, wherein the air-permeable, non-woven structure is compostable in accordance with EN 13432 and or ASTM D6400.

19. A method as claimed in any of the preceding claims, wherein the absorbent particles are hydrophilic.

20. A method as claimed in any of the preceding claims wherein the resulting air-permeable, non-woven structure and absorbent particle matrix is further consolidated by the application of heat and/or pressure.

21. A method as claimed in any of the preceding claims wherein the resulting air-permeable, non-woven structure and absorbent particle matrix is subjected to a heated through air process to consolidate the fibres by partial melting and simultaneously to attach said partially melted fibres to said incorporated absorbent particles, thereby to prevent diffusion of the particles from the air-permeable structure.

22. A method as claimed in any of the preceding claims wherein, in step (ii) the dispersion of the absorbent particles is made across the entire surface of said air-permeable, non-woven structure.

23. A method as claimed in any of claims 1 to 21 wherein, in step (ii) the dispersion of the absorbent particles is made across selected specific areas of the surface of said air-permeable, non-woven structure.

24. A method as claimed in claim 23, further comprising the step, after at least the dispersion (ii) and dissipation (iii) steps, of cutting or otherwise extracting said selected specific areas from the surrounding air permeable substrate.

25. A method as claimed in claim 24, further comprising the subsequent step of welding or sealing the edges of said extracted selected specific areas on a line on or within 30 millimetres of the cut line.
